(19) United States
(12) Patent Application Publication
(10) Pub. No.: US 2026/0345509 A1
(43) Pub. Date: Nov. 5, 2026

(54) DONEPEZIL SUSTAINED-RELEASE MICROSPHERE, PREPARATION METHOD THEREFOR, PHARMACEUTICAL FORMULATION, AND USE THEREOF

(71) Applicant: HUILLY PHARMA CO., LTD, Suzhou (CN)

(72) Inventors: Hang Wang, Suzhou, Jiangsu (CN); Jie Zhu, Suzhou, Jiangsu (CN); Wenwen Liang, Suzhou, Jiangsu (CN); Chun Zhang, Suzhou, Jiangsu (CN); Ping Xu, Suzhou, Jiangsu (CN)

(73) Assignee: HUILLY PHARMA CO., LTD, Suzhou (CN)

(21) Appl. No.: 19/706,231

(22) Filed: Jun. 12, 2026

Related U.S. Application Data
(63) Continuation of application No. PCT/CN2024/128377, filed on Oct. 30, 2024.

(30) Foreign Application Priority Data
Jan. 2, 2024 (CN) 2024100041109

(51) Int. Cl. A61K 9/51; A61J 3/00; A61K 31/445

(52) U.S. Cl. CPC A61K 9/5153; A61J 3/00; A61K 9/5192; A61K 31/445

(57) ABSTRACT

Provided are a donepezil sustained-release microsphere, a preparation method therefor, a pharmaceutical preparation thereof, and use thereof. The preparation method includes the following steps: forming an aqueous phase (including a surfactant, an osmotic pressure regulator, and water, and adjusting a pH value with a pH regulator); forming an oil phase (including donepezil, a carrier material, and an organic solvent); adding the oil phase to the aqueous phase for dispersion to form a pre-emulsion, then carrying out membrane emulsification on the pre-emulsion to form an emulsion; and finally curing, washing, and lyophilizing the emulsion to obtain the donepezil sustained-release microsphere. The preparation process is stable and controllable, with good batch-to-batch repeatability. The microspheres have a smooth and intact surface, uniform particle size distribution and controllable particle size, and possess high drug loading capacity. The microspheres enable long-term and sustained drug release, and exhibit excellent injectability, so that an administration frequency can be reduced, and adverse gastrointestinal reactions can be alleviated.

### CROSS-REFERENCE TO RELATED APPLICATION

This patent application is a Continuation of PCT Patent Application No. PCT/CN2024/128377 filed on Oct. 30, 2024, which claims priority to Chinese Patent Application No. 202410004110.9, filed with the China National Intellectual Property Administration on Jan. 2, 2024 and entitled “Donepezil sustained-release microsphere, preparation method therefor, pharmaceutical formulation, and use thereof”, which is incorporated herein by reference in its entirety.

### TECHNICAL FIELD

The present disclosure belongs to the field of pharmaceutical formulations, and in particular to a donepezil sustained-release microsphere, a preparation method therefor, a pharmaceutical formulation, and use thereof.

### BACKGROUND

Alzheimer's disease (AD) is a chronic neurodegenerative disease, typically with onset in presenile and senile periods, which is characterized by progressive memory impairment, cognitive dysfunction, personality changes, and language disorders, severely affecting social, occupational, and daily living functions.

As the pathogenesis of Alzheimer's disease is highly complex, the current pharmacological treatments and other intervention approaches cannot radically cure Alzheimer's disease. The mechanism of action of most drug treatments is to regulate some neurotransmitters in the brain, primarily to alleviate symptoms and slow disease progression, resulting in limited therapeutic effects.

Dysfunction of cortical cholinergic neurotransmitters is regarded as one of causes of memory impairment and other cognitive dysfunctions. Currently approved agents for the treatment of Alzheimer's disease (AD) are an acetylcholinesterase (AChE) inhibitor and an N-methyl-D-aspartate (NMDA) receptor antagonist. Donepezil is a second-generation central acetylcholinesterase inhibitor (AChEI), featuring characteristics of high pharmacological activity, strong selectivity, and favorable safety profile. With high lipophilicity, donepezil can readily penetrate a blood-brain barrier, has high affinity for acetylcholinesterase, specifically binds to cholinesterase in the cerebral cortex, and inhibits cholinesterase activity, yielding high efficacy and few adverse reactions. Therefore, the donepezil is a first-line clinical drug for the treatment of mild to moderate Alzheimer's disease.

At present, most commercially available formulations are oral donepezil hydrochloride formulations, including oral tablets, capsules, oral solutions, and the like, which need to be administered once a day, with a course of treatment lasting as long as 3 to 6 months. However, patients with Alzheimer's disease suffer from varying degrees of memory impairment, which makes them prone to missed doses, incorrect administration, and even medication refusal. In addition, when the disease progresses to a certain extent, patients may also experience dysphagia, all of these problems are not conducive to improving the condition. In addition, oral administration of donepezil hydrochloride is associated with some adverse reactions, such as nausea, vomiting, diarrhea, and anorexia. The control of Alzheimer's disease requires long-term maintenance, while long-term oral medication may lead to poor patient compliance and low medication accuracy.

The membrane emulsification method enables the preparation of microspheres with uniform and controllable particle size, high encapsulation efficiency, and low mechanical strength. Due to differences in drug indications and administration routes, in vivo environment where microspheres are located also varies. Therefore, different requirements are put forward for the types, particle sizes, and distribution of drug-loaded microspheres, and parameter regulation, condition optimization and improvement of related equipment in membrane emulsification operation need to be strictly controlled. For example, both highly hydrophilic polypeptide drugs and low-molecular-weight drugs are prone to drug escape, but regulation of factors such as membrane pore sizes, types, and relative proportions of organic solvents, and stirring speed enable the microspheres to achieve excellent encapsulation efficiency and drug loading capacity. In addition, researchers have also found that during use of the membrane emulsification technology, this operation process involves multidisciplinary knowledge and numerous influencing factors, and there is currently no complete set of theories to guide this preparation technology.

### SUMMARY

### Problem to be Solved by the Present Disclosure

In view of the foregoing problem, the present disclosure is intended to provide a donepezil sustained-release microsphere formulation with uniform particle size, high drug loading capacity, long-term stable drug release, excellent injectability, stable and controllable preparation process, and excellent batch-to-batch repeatability.

### Solution for Solving the Problem

According to a first aspect, the present disclosure provides a preparation method for a donepezil sustained-release microsphere, including the following steps:
mixing a surfactant, an osmotic pressure regulator, and water to obtain a mixture, adjusting a pH value of the mixture with a pH regulator to form an aqueous phase;
mixing donepezil, a carrier material, and an organic solvent to form an oil phase; and
dispersing the oil phase in the aqueous phase to form a pre-emulsion, carrying out membrane emulsification on the pre-emulsion to form an emulsion; finally solidifying, washing, and lyophilizing the emulsion to obtain the donepezil sustained-release microsphere.

As a further improvement of the present disclosure, the pre-emulsion is an oil-in-water pre-emulsion.

As a further improvement of the present disclosure, the surfactant is at least one of polyvinyl alcohol (PVA), Tween, sodium dodecyl sulfonate (SDS), sodium dodecyl benzene sulfonate (SDBS), and polyoxyethylene octylphenol ether, preferably polyvinyl alcohol.

As a further improvement of the present disclosure, a weight ratio of the surfactant to donepezil is from 1:5 to 20:5, preferably from 4:5 to 8:5.

As a further improvement of the present disclosure, the osmotic pressure regulator is at least one of sodium chloride and potassium chloride, preferably sodium chloride.

As a further improvement of the present disclosure, a weight ratio of the osmotic pressure regulator to donepezil is from 1:5 to 6:5, preferably from 1.5:5 to 4:5.

As a further improvement of the present disclosure, the water is at least one of ultra-pure water, double-distilled water, and water for injection, preferably ultra-pure water.

As a further improvement of the present disclosure, a volume ratio of the water to the organic solvent is from 5:1 to 50:1, preferably from 8:1 to 14:1.

As a further improvement of the present disclosure, the pH regulator is at least one of hydrogen chloride or sodium hydroxide.

As a further improvement of the present disclosure, the pH regulator is used to adjust a pH value of the mixture to from 6.0 to 12.0, preferably from 6.0 to 10.0.

As a further improvement of the present disclosure, the carrier material is a biodegradable carrier material.

As a further improvement of the present disclosure, the carrier material is at least one of poly(lactic-co-glycolic acid) (PLGA), poly(lactic acid)-poly(ethylene glycol) copolymer (PELA), and polylactic acid (PLA), preferably poly(lactic-co-glycolic acid).

As a further improvement of the present disclosure, the carrier material has intrinsic viscosity from 0.15 dL/g to 0.90 dL/g, preferably from 0.4 dL/g to 0.7 dL/g.

As a further improvement of the present disclosure, a weight ratio of the carrier material to donepezil is from 2:3 to 12:3, preferably from 2:3 to 5:3.

As a further improvement of the present disclosure, the organic solvent is at least one of trichloromethane, dichloromethane, ethyl acetate, acetone, acetonitrile, and absolute ethyl alcohol, preferably dichloromethane.

As a further improvement of the present disclosure, a dosage ratio of the organic solvent to donepezil is from 4 to 20 mL:1 g, preferably from 6 to 15 mL:1 g.

As a further improvement of the present disclosure, the dispersion is accomplished by means of at least one of stirring and ultrasonication, preferably stirring, more preferably mechanical stirring.

As a further improvement of the present disclosure, the mechanical stirring lasts for 30 to 60 s, preferably 30 s.

As a further improvement of the present disclosure, the mechanical stirring is carried out at a rotational speed of 250 to 350 rpm, preferably 300 rpm.

As a further improvement of the present disclosure, the emulsification is accomplished by means of membrane emulsification.

As a further improvement of the present disclosure, the solidification is accomplished by means of volatilization or evaporation, preferably reduced-pressure volatilization, stirring-based volatilization, or thin-film evaporation, more preferably reduced-pressure volatilization.

As a further improvement of the present disclosure, the reduced-pressure volatilization is carried out at a vacuum degree of −0.09 to −0.05 MPa, preferably −0.09 MPa.

As a further improvement of the present disclosure, the reduced-pressure volatilization lasts for 20 to 300 min, preferably 20 to 240 min.

As a further improvement of the present disclosure, the reduced-pressure volatilization is carried out at a temperature of 20 to 42 °C, preferably 38 °C.

As a further improvement of the present disclosure, the preparation method further includes solidifying the emulsion, followed by washing and lyophilizing.

According to a second aspect, the present disclosure provides a donepezil sustained-release microsphere, which is prepared by using the foregoing preparation method.

As a further improvement of the present disclosure, the donepezil sustained-release microsphere has a particle size of 10 to 50 μm, preferably 25 to 35 μm.

As a further improvement of the present disclosure, encapsulation efficiency of the donepezil sustained-release microsphere is over 80%, preferably over 90%.

As a further improvement of the present disclosure, a drug loading capacity of the donepezil sustained-release microsphere is 20% to 60%, preferably 40% to 60%.

According to a third aspect, the present disclosure provides a pharmaceutical formulation of donepezil, including the foregoing sustained-release microsphere.

As a further improvement of the present disclosure, the pharmaceutical formulation further includes at least one pharmaceutically acceptable excipient.

As a further improvement of the present disclosure, the pharmaceutical formulation is a sustained-release formulation.

As a further improvement of the present disclosure, the pharmaceutical formulation is an injection.

According to a fourth aspect, the present disclosure provides use of the sustained-release microsphere described above or the pharmaceutical formulation described above in preparation of a drug for preventing and/or treating a disease and/or disorder at least partially associated with acetylcholinesterase.

As a further improvement of the present disclosure, the disease and/or disorder at least partially associated with acetylcholinesterase is Alzheimer's disease.

### Beneficial Effects of the Present Disclosure

The present disclosure has beneficial effects as follows.

(1) The preparation method provided by the present disclosure can minimize drug escape and avoid a risk of high initial burst release.

(2) The sustained-release microsphere prepared by the present disclosure is loaded with high-content donepezil, the dosage of the microspheres is relatively less during dosing, thereby avoiding an injection nodule or other adverse reactions caused by excessively large injection volume.

(3) The microspheres prepared by the present disclosure have uniform particle size and excellent injectability.

(4) The preparation process provided by the present disclosure is stable, controllable, and has outstanding economic benefits and broad application prospects.

(5) The long-acting sustained-release microsphere formulation prepared by the present disclosure can greatly reduce a dosing frequency of the patient, thereby effectively avoiding fluctuations in plasma concentration caused by incorrect or missed administration, as well as subsequent recurrence or deterioration of the disease.

(6) A microsphere formulation for injection prepared by the present disclosure can effectively avoid gastrointestinal adverse reactions, and has significant clinical advantages.

### BRIEF DESCRIPTION OF THE DRAWINGS

FIG. 1 is a scanning electron micrograph of a microsphere prepared according to Embodiment 1;

FIG. 2 is a scanning electron micrograph of a microsphere prepared according to Embodiment 2;

FIG. 3 is a scanning electron micrograph of a microsphere prepared according to Embodiment 3;

FIG. 4 is a scanning electron micrograph of a microsphere prepared according to Embodiment 4;

FIG. 5 is a scanning electron micrograph of a microsphere prepared according to Embodiment 5;

FIG. 6 is a scanning electron micrograph of a microsphere prepared according to Embodiment 6;

FIG. 7 is a scanning electron micrograph of a microsphere prepared according to Embodiment 7;

FIG. 8 is a scanning electron micrograph of a microsphere prepared according to Embodiment 8;

FIG. 9 is a scanning electron micrograph of a microsphere prepared according to Comparative Example 1;

FIG. 10 is a scanning electron micrograph of a microsphere prepared according to Comparative Example 2;

FIG. 11 shows in vitro cumulative release curves of five types of microspheres prepared according to Embodiments 1, 5, 6, 7, and 8.

### DETAILED DESCRIPTION OF THE EMBODIMENTS

Various exemplary embodiments, features, and aspects of the present disclosure are described below in detail. As used herein, a term “exemplary” means “serving as an example, an embodiment, or an illustration”. Any embodiment described herein as “exemplary” shall not be construed as superior or preferable to other embodiments.

In addition, to better illustrate the present disclosure, numerous specific details are given out in the following specific implementations. Those skilled in the art shall understand that the present disclosure may also be implemented without some specific details. In some other instances, methods, means, devices, and steps well known to those skilled in the art are not described in detail, thereby highlighting the gist of the present disclosure.

Unless otherwise specified, all units used in this specification are international standard units. All values and numerical ranges in the present disclosure shall be understood to include systematic errors unavoidable in industrial production.

In this specification, the term “may” includes both possibilities of carrying out a specific treatment and omitting such a treatment.

In this specification, references to “some specific/preferred implementations”, “other specific/preferred implementations”, “implementations”, and the like mean that specific elements (for example, features, structures, properties, and/or characteristics) described in connection with such implementations are included in at least one implementation described herein, and may or may not be present in other implementations. In addition, it should be understood the elements may be combined in any appropriate manner in a variety of implementations.

In this specification, a numerical range expressed in the form of “value A to value B” shall include endpoint values A and B.

In this specification, when terms “normal temperature” and “room temperature” are used, the temperature may range from 10 to 40 °C.

### Donepezil Sustained-Release Microsphere and Preparation Method Therefor

The present disclosure provides a preparation method for a donepezil sustained-release microsphere, including the following steps:

Mix a surfactant, an osmotic pressure regulator, and water to obtain a mixture, adjust a pH value of the mixture with a pH regulator to form an aqueous phase.

Mix donepezil, a carrier material, and an organic solvent to form an oil phase.

Disperse the oil phase in the aqueous phase to form a pre-emulsion, carry out membrane emulsification on the pre-emulsion to form an emulsion; finally solidify, wash, and lyophilize the emulsion to obtain the donepezil sustained-release microsphere.

During preparation, the donepezil diffuses from the oil phase into the aqueous phase due to osmotic pressure and isoelectric point factors, causing a drug to migrate to the outside of the carrier material. This results in an excessively fast release of the microsphere at an initial release stage, making it difficult to achieve long-term stable release. In addition to that, the drug migration may form drug crystallization at a periphery or surface of the microsphere, which roughens the surface of the microsphere and reduces a drug loading capacity. In addition, the particle size of the microsphere significantly affects a drug release behavior, but the microsphere prepared by a conventional preparation method is nonuniform in particle size distribution, which may affect stable release of the drug. Therefore, avoiding drug escape and controlling microsphere size are particularly important during preparation.

According to the present disclosure, a specific amount of osmotic pressure regulator is added to the aqueous phase and a pH value of the aqueous phase is regulated, thereby preventing the drug from migrating to the outside of the microsphere. This helps the microsphere have smoother surface and higher drug loading capacity, thereby effectively avoiding burst release of the drug and maintain stable release behavior of the drug. In addition, an emulsion with more uniform particle size can be obtained by means of a membrane emulsification method, no additional sieving is required during washing and collection of microspheres, the particle size of the microsphere is controllable, so that drug loss can be effectively avoided, long-term stable release of the drug can be achieved.

In an implementation of the present disclosure, the pre-emulsion in the foregoing preparation method may be an oil-in-water pre-emulsion.

In an implementation of the present disclosure, a surfactant in the foregoing preparation method may be at least one of polyvinyl alcohol, Tween, sodium dodecyl sulfonate, sodium dodecyl benzene sulfonate, and polyoxyethylene octylphenol ether, such as polyvinyl alcohol, Tween, or a mixture thereof in any mixing ratio.

In an implementation of the present disclosure, the surfactant in the foregoing preparation method may be polyvinyl alcohol.

In an implementation of the present disclosure, a weight ratio of the surfactant in the foregoing preparation method to donepezil may vary within a specific range, for example 1:5 to 5:20.

In an implementation of the present disclosure, the weight ratio of the surfactant in the foregoing preparation method to donepezil may be from 4:5 to 8:5.

In an implementation of the present disclosure, an osmotic pressure regulator in the foregoing preparation method may be at least one of sodium chloride and potassium chloride, such as sodium chloride, potassium chloride, or a mixture thereof in any mixing ratio.

In an implementation of the present disclosure, the osmotic pressure regulator in the foregoing preparation method may be sodium chloride.

In an implementation of the present disclosure, a weight ratio of the osmotic pressure regulator in the foregoing preparation method to donepezil may vary within a specific range, for example from 1:5 to 6:5.

In an implementation of the present disclosure, the weight ratio of the osmotic pressure regulator in the foregoing preparation method to donepezil may be from 1.5:5 to 4:5.

In an implementation of the present disclosure, the water in the foregoing preparation method may be at least one of ultra-pure water, double-distilled water, and water for injection, such as ultra-pure water, water for injection, or a mixture thereof in any mixing ratio.

In an implementation of the present disclosure, the water in the foregoing preparation method may be ultra-pure water.

In an implementation of the present disclosure, a volume ratio of the water in the foregoing preparation method to the organic solvent may vary within a specific range, for example from 5:1 to 50:1.

In an implementation of the present disclosure, a weight ratio of the water in the foregoing preparation method to donepezil may be from 8:1 to 14:1.

In an implementation of the present disclosure, the pH regulator in the foregoing preparation method may be at least one of hydrogen chloride and sodium hydroxide.

In an implementation of the present disclosure, the pH regulator in the foregoing preparation method may be used to adjust a pH value of the mixture within a specific range, for example from 6.0 to 12.0.

In an implementation of the present disclosure, the pH regulator in the foregoing preparation method may be used to adjust the pH value of the mixture to from 6.0 to 10.0.

In an implementation of the present disclosure, the carrier material in the foregoing preparation method may be a biodegradable carrier material.

In an implementation of the present disclosure, the carrier material in the foregoing preparation method may be at least one of at least one of poly(lactic-co-glycolic acid), poly(lactic acid)-poly(ethylene glycol) copolymer, and polylactic acid, such as poly(lactic-co-glycolic acid), poly(lactic acid)-poly(ethylene glycol) copolymer, or a mixture thereof in any mixing ratio.

In an implementation of the present disclosure, the carrier material in the foregoing preparation method may be poly(lactic-co-glycolic acid).

In an implementation of the present disclosure, intrinsic viscosity of the carrier material in the foregoing preparation method may be from 0.15 dL/g to 0.90 dL/g, for example, the carrier material in the foregoing preparation method may be poly(lactic-co-glycolic acid) with intrinsic viscosity from 0.15 dL/g to 0.90 dL/g.

In an implementation of the present disclosure, intrinsic viscosity of the carrier material in the preparation method may be from 0.4 dL/g to 0.7 dL/g, for example, the carrier material in the foregoing preparation method may be poly(lactic-co-glycolic acid) with intrinsic viscosity from 0.4 dL/g to 0.7 dL/g.

In an implementation of the present disclosure, a weight ratio of the carrier material in the foregoing preparation method to donepezil may vary within a specific range, for example from 2:3 to 12:3.

In an implementation of the present disclosure, the weight ratio of the carrier material in the foregoing preparation method to donepezil may be from 2:3 to 5:3.

In an implementation of the present disclosure, the organic solvent in the foregoing preparation method may be at least one of trichloromethane, dichloromethane, ethyl acetate, acetone, acetonitrile, and absolute ethyl alcohol, such as dichloromethane, ethyl acetate, or a mixture thereof in any mixing ratio.

In an implementation of the present disclosure, the organic solvent in the foregoing preparation method may be dichloromethane.

In an implementation of the present disclosure, a dosage ratio of the organic solvent in the foregoing preparation method to donepezil may vary within a specific range, for example from 4 to 20 mL:1 g.

In an implementation of the present disclosure, the dosage ratio of the organic solvent in the foregoing preparation method to donepezil may be from 6 to 15 mL:1 g.

In an implementation of the present disclosure, dispersion in the foregoing preparation method may be accomplished by means of at least one of stirring and ultrasonication.

In an implementation of the present disclosure, the dispersion in the foregoing preparation method may be accomplished by means of stirring.

In an implementation of the present disclosure, the dispersion in the foregoing preparation method may be accomplished by means of mechanical stirring.

In an implementation of the present disclosure, a duration of the mechanical stirring in the foregoing preparation method may vary within a specific range, for example 30 to 60 s.

In an implementation of the present disclosure, the duration of the mechanical stirring in the foregoing preparation method may be 30 s.

In an implementation of the present disclosure, a rotational speed of the mechanical stirring in the foregoing preparation method may vary within a specific range, for example 250 to 350 rpm.

In an implementation of the present disclosure, the rotational speed of the mechanical stirring in the foregoing preparation method may be 300 rpm.

In an implementation of the present disclosure, emulsification in the foregoing preparation method may be accomplished by means of membrane emulsification.

In an implementation of the present disclosure, solidification in the foregoing preparation method may be accomplished by means of volatilization or evaporation.

In an implementation of the present disclosure, the solidification in the foregoing preparation method may be accomplished by means of pressure-reduced volatilization, stirring-based volatilization, or thin-film evaporation.

In an implementation of the present disclosure, the solidification in the foregoing preparation method may be accomplished by means of pressure-reduced volatilization.

In an implementation of the present disclosure, a vacuum degree of the pressure-reduced volatilization in the foregoing preparation method may vary within a specific range, for example −0.09 to −0.05 MPa.

In an implementation of the present disclosure, the vacuum degree of the pressure-reduced volatilization in the foregoing preparation method may be −0.09 MPa.

In an implementation of the present disclosure, a duration of the pressure-reduced volatilization in the foregoing preparation method may vary within a specific range, for example 20 to 300 min.

In an implementation of the present disclosure, the duration of the pressure-reduced volatilization in the foregoing preparation method may be from 20 to 240 min.

In an implementation of the present disclosure, a temperature of the pressure-reduced volatilization in the foregoing preparation method may vary within a specific range, for example 20 to 42 °C.

In an implementation of the present disclosure, the temperature of the pressure-reduced volatilization in the foregoing preparation method may be 38 °C.

In an implementation of the present disclosure, the preparation method may further include the following steps: solidify the emulsion, followed by washing and lyophilizing.

Correspondingly, the present disclosure provides a donepezil sustained-release microsphere, which is prepared by using the foregoing preparation method.

In an implementation of the present disclosure, a particle size of the donepezil sustained-release microsphere prepared by using the foregoing preparation method may be 10 to 50 μm.

In an implementation of the present disclosure, a particle size of the donepezil sustained-release microsphere prepared by using the foregoing preparation method may be 25 to 35 μm.

In an implementation of the present disclosure, encapsulation efficiency of the donepezil sustained-release microsphere prepared by using the foregoing preparation method may be over 80%.

In an implementation of the present disclosure, the encapsulation efficiency of the donepezil sustained-release microsphere prepared by using the foregoing preparation method may be over 90%.

In an implementation of the present disclosure, a drug loading capacity of the donepezil sustained-release microsphere prepared by using the foregoing preparation method may be 20% to 60%.

In an implementation of the present disclosure, the drug loading capacity of the donepezil sustained-release microsphere prepared by using the foregoing preparation method may be 40% to 60%.

### Pharmaceutical Formulation of Donepezil

The present disclosure provides a pharmaceutical formulation of donepezil, including the foregoing sustained-release microsphere.

In an implementation of the present disclosure, the pharmaceutical formulation may further include at least one pharmaceutically acceptable excipient, such as a solvent, a solubilizer, and a cosolvent.

In an implementation of the present disclosure, the pharmaceutical formulation may be a formulation with a specific drug release behavior, such as a sustained-release formulation.

In an implementation of the present disclosure, the pharmaceutical formulation may be a parenteral dosage form, such as an injection.

### Medical Use of Donepezil Sustained-Release Microsphere and Pharmaceutical Formulation Thereof

The present disclosure provides use of the sustained-release microsphere described above or the pharmaceutical formulation described above in preparation of a drug for preventing and/or treating a disease and/or disorder at least partially associated with acetylcholinesterase.

In an implementation of the present disclosure, the disease and/or disorder at least partially associated with acetylcholinesterase is Alzheimer's disease.

### Method for Determining Parameters of Donepezil Sustained-Release Microsphere

A particle size and particle size distribution determination method is used to determine a particle size and span value of the microsphere (particle size distribution span, the smaller the span value, the more uniform the particle size of the microspheres), with details as follows:

Add 50 mg of microsphere to 20 mL of water, shake and ultrasonicate for 10 min to uniformly disperse the microsphere to obtain a solution, then transfer the solution to a sample cell of a laser particle size analyzer, and determine a particle size and calculate a span value, with a calculation formula as follows:

$$Span = \frac{{D}_{v , 90 \%} - {D}_{v , 10 \%}}{{D}_{v , 50 \%}}$$

where D_{v,90%}, D_{v,50%}, and D_{v,10%} are corresponding particle sizes of the microsphere when volume fractions are 90%, 50%, and 10%, respectively.

A high-performance liquid chromatography is used to detect a drug loading capacity and encapsulation efficiency of the microsphere, with details as follows.

A method for detecting the drug loading capacity is as follows: dissolve 25 mg of microsphere completely in 80% v/v acetonitrile to obtain a solution, then dilute the solution to 50 mL, pipette 1 mL of the solution and further dilute the same to 10 mL, inject 20 μL of the diluted solution into an HPLC (High-Performance Liquid Chromatography) system (Welch Ultimate XB-C18, 150×4.6 mm, 5 μm), measure at a detection wavelength of 271 nm, where a used mobile phase is prepared by mixing phosphate buffer (pH 5.0) and acetonitrile in a ratio of 7:3.

A method for detecting the encapsulation efficiency is as follows: wash 25 mg of microsphere with a PBS (Phosphate-Buffered Saline) solution, dissolve a solid completely in 80% v/v acetonitrile to obtain a solution, dilute the solution to 50 mL, and then pipette 1 mL of the solution and further dilute the same to 10 mL, with subsequent operations the same as those for the method for detecting the drug loading capacity.

Calculation formulas of the drug loading capacity and the encapsulation efficiency are as follows:

$$Drug loading capacity = \frac{the mass of donepezil in the microsphere}{the total mass of donepezil} \times 100 \%$$

$$Encapsulation efficiency = \frac{the drug amount encapsulated in the microsphere}{the total drug amount encapsulated and not encapsulated in the microsphere} \times 100 \%$$

### EMBODIMENT

Implementations of the present disclosure are described in detail below with reference to embodiments, but those skilled in the art should understand that the following embodiments are merely used to illustrate the present disclosure and should not be regarded as limiting the scope of the present disclosure. For specific conditions not specified in the embodiments, the conventional conditions or the conditions suggested by the manufacturer shall be followed. All reagents and instruments used without specifying the manufacturer are standard laboratory products that are commercially available.

### Embodiment 1

1.5 g of donepezil and 1.0 g of PLGA (LA:GA=75:25, intrinsic viscosity is from 0.4 to 0.5 dL/g) are weighed and then dissolved in 10 mL of dichloromethane to prepare O. 100 mL of polyvinyl alcohol aqueous solution with a concentration of 1.0% is prepared to serve as an outer aqueous phase, and then W is prepared without pH adjustment. O is added to W, and mechanical stirring is carried out at a rotational speed of 300 rpm for 30 s to prepare a pre-emulsion. The pre-emulsion is poured into a rapid membrane emulsifier for emulsification and dispersion, where a used membrane tube has a pore size of 20 μm. The emulsion is solidified at a vacuum degree of −0.09 MPa and a temperature of 38 °C for 60 min. After the solidification is finished, washing and lyophilizing are carried out to obtain a donepezil sustained-release microsphere.

A particle size and particle size distribution determination method is used to determine that the prepared sustained-released microspheres have an average particle size of 13 μm and a Span value of 1.2. A high-performance liquid chromatography is used to determine a drug loading capacity and encapsulation efficiency of the prepared sustained-released microspheres, where the drug loading capacity is 49.5%, and the encapsulation efficiency is 82.5%.

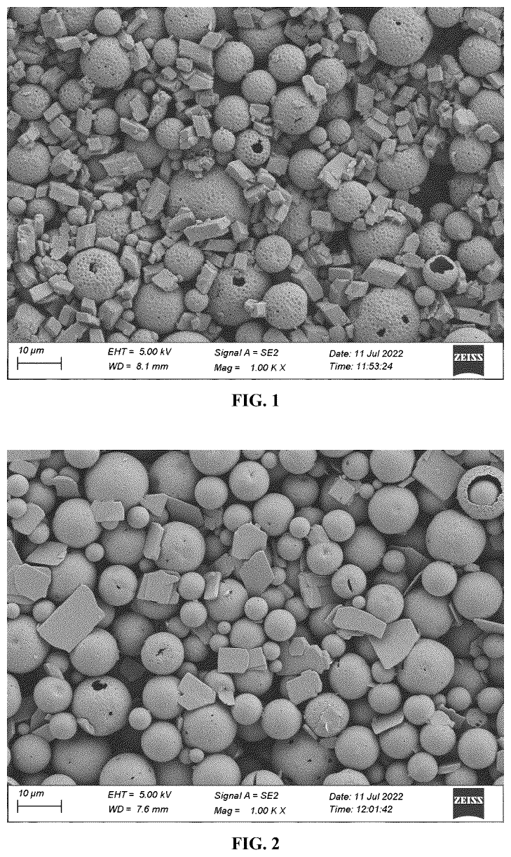

It can be seen from FIG. 1 that the donepezil sustained-release microspheres prepared in Embodiment 1 exhibit extensive breakage, non-uniform particle size, rough surface morphology, severe collapse, and a large number of tiny pores, with drug escape and precipitation resulting in the formation of flake-like or block-like crystals.

In the prior art, it is proposed to use an ice water bath during preparation of the pre-emulsion to minimize drug escape. However, the temperature may affect viscosity of the dispersed phase and a continuous phase, solubility of the surfactant, a phase transition temperature, and diffusion performance, as well as interfacial tension between the two phases, thereby further affecting the particle size and performance of the microspheres. The preparation provided by the present disclosure is carried out without low-temperature treatment, and the obtained microspheres feature uniform particle size and stable drug release.

### Embodiment 2

1.5 g of donepezil and 1.0 g of PLGA (LA:GA=75:25, intrinsic viscosity is from 0.4 to 0.5 dL/g) are weighed and then dissolved in 10 mL of dichloromethane to prepare O. 100 mL of polyvinyl alcohol aqueous solution with a concentration of 1.0% is prepared to serve as an outer aqueous phase, then 1.0 g of sodium chloride is added to prepare W without pH adjustment. O is added to W, and mechanical stirring is carried out at a rotational speed of 300 rpm for 30 s to prepare a pre-emulsion. The pre-emulsion is poured into a rapid membrane emulsifier for emulsification and dispersion, where a used membrane tube has a pore size of 20 μm. The emulsion is solidified at a vacuum degree of −0.09 MPa and a temperature of 38 °C for 60 min. After the solidification is finished, washing and lyophilizing are carried out to obtain a donepezil sustained-release microsphere.

A particle size and particle size distribution determination method is used to determine that the prepared sustained-released microspheres have an average particle size of 12 μm and a Span value of 1.0. A high-performance liquid chromatography is used to determine a drug loading capacity and encapsulation efficiency of the prepared sustained-released microspheres, where the drug loading capacity is 51.1%, and the encapsulation efficiency is 85.17%.

By comparing FIG. 1 and FIG. 2, it can be learned that after a specific amount of osmotic pressure regulator to the aqueous phase in Embodiment 2, although the donepezil sustained-release microspheres still have damaged pores and severe collapse, and there are still flake-like or block-like crystals formed by drug escape and precipitation on the exterior of the microspheres, the surface of the microsphere is relatively smooth and intact. This indicates that the addition of the osmotic pressure regulator in the present disclosure can inhibit drug escape, making the surface of the microsphere smoother.

### Embodiment 3

1.5 g of donepezil and 1.0 g of PLGA (LA:GA=75:25, intrinsic viscosity is from 0.4 to 0.5 dL/g) are weighed and then dissolved in 10 mL of dichloromethane to prepare O. 100 mL of polyvinyl alcohol aqueous solution with a concentration of 1.0% is prepared to serve as an outer aqueous phase, a pH value of the solution is adjusted to 6.0 by sodium hydroxide to prepare W. O is added to W, and mechanical stirring is carried out at a rotational speed of 300 rpm for 30 s to prepare a pre-emulsion. The pre-emulsion is poured into a rapid membrane emulsifier for emulsification and dispersion, where a used membrane tube has a pore size of 20 μm. The emulsion is solidified at a vacuum degree of −0.09 MPa and a temperature of 38 °C for 60 min. After the solidification is finished, washing and lyophilizing are carried out to obtain a donepezil sustained-release microsphere.

A particle size and particle size distribution determination method is used to determine that the prepared sustained-released microspheres have an average particle size of 13 μm and a Span value of 0.7. A high-performance liquid chromatography is used to determine a drug loading capacity and encapsulation efficiency of the prepared sustained-released microspheres, where the drug loading capacity is 55.2%, and the encapsulation efficiency is 92.0%.

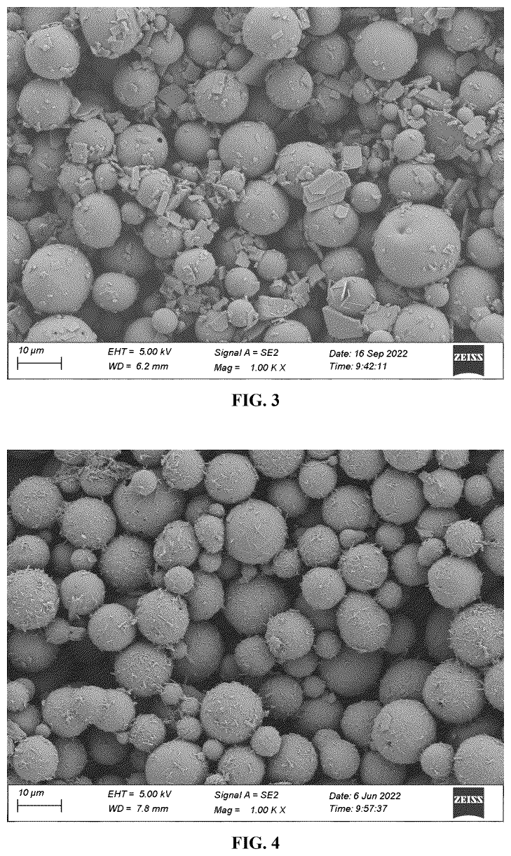

By comparing FIG. 1 and FIG. 2, it can be learned that the pH value is adjusted to 8 in Embodiment 3. In FIG. 3, although the donepezil sustained-release microspheres still show a large amount of precipitated drug crystals, the surface of the microsphere is relatively smooth and intact, without apparent surface damage or large pores. This indicates that the addition of the pH regulator can effectively improve the breakage of the microspheres, making the surface of the microsphere smoother.

### Embodiment 4

1.5 g of donepezil and 1.0 g of PLGA (LA:GA=75:25, intrinsic viscosity is from 0.4 to 0.5 dL/g) are weighed and then dissolved in 10 mL of dichloromethane to prepare O. 100 mL of polyvinyl alcohol aqueous solution with a concentration of 1.0% is prepared to serve as an outer aqueous phase, a pH value of the solution is adjusted to 10.0 by sodium hydroxide to prepare W. O is added to W, and mechanical stirring is carried out at a rotational speed of 300 rpm for 30 s to prepare a pre-emulsion. The pre-emulsion is poured into a rapid membrane emulsifier for emulsification and dispersion, where a used membrane tube has a pore size of 20 μm. The emulsion is solidified at a vacuum degree of −0.09 MPa and a temperature of 38 °C for 60 min. After the solidification is finished, washing and lyophilizing are carried out to obtain a donepezil sustained-release microsphere.

A particle size and particle size distribution determination method is used to determine that the prepared sustained-released microspheres have an average particle size of 13 μm and a Span value of 0.7. A high-performance liquid chromatography is used to determine a drug loading capacity and encapsulation efficiency of the prepared sustained-released microspheres, where the drug loading capacity is 57.5%, and the encapsulation efficiency is 95.8%.

By comparing FIG. 1, FIG. 3, and FIG. 4, it can be learned that although there are still some crystals on the donepezil sustained-release microspheres prepared in Embodiment 4, these crystals are attached the surface of the microsphere in fine strip or filamentous forms, without escaping extensively to form block-like or flake-like aggregates, and without apparent surface damage or large pores. This indicates that the addition of the pH regulator and maintenance of a specific alkali environment can effectively reduce crystal escape, and improve the phenomenon of surface damage and large pores on the microspheres.

### Embodiment 5

3.0 g of donepezil and 2.0 g of PLGA (LA:GA=50:50, intrinsic viscosity is from 0.6 to 0.7 dL/g) are weighed and then dissolved in 20 mL of dichloromethane to prepare O. 200 mL of polyvinyl alcohol aqueous solution with a concentration of 1.0% is prepared to serve as an outer aqueous phase, a pH value of the solution is adjusted to 10.0 by sodium hydroxide to prepare W. O is added to W, and mechanical stirring is carried out at a rotational speed of 300 rpm for 30 s to prepare a pre-emulsion. The pre-emulsion is poured into a rapid membrane emulsifier for emulsification and dispersion, where a used membrane tube has a pore size of 20 μm. The emulsion is solidified at a vacuum degree of −0.09 MPa and a temperature of 38 °C for 60 min. After the solidification is finished, washing and lyophilizing are carried out to obtain a donepezil sustained-release microsphere.

A particle size and particle size distribution determination method is used to determine that the prepared sustained-released microspheres have an average particle size of 14 μm and a Span value of 0.7. A high-performance liquid chromatography is used to determine a drug loading capacity and encapsulation efficiency of the prepared sustained-released microspheres, where the drug loading capacity is 56.2%, and the encapsulation efficiency is 93.4%.

It can be seen from FIG. 5 that there are still some small flake-type crystals on the exterior of the donepezil sustained-release microspheres of Embodiment 5, and the surface of the microsphere is relatively smooth, intact, and free of damage.

### Embodiment 6

6 g of donepezil and 4 g of PLGA (LA:GA=50:50, intrinsic viscosity is from 0.6 to 0.7 dL/g) are weighed and then dissolved in 40 mL of dichloromethane to prepare O. 400 mL of polyvinyl alcohol aqueous solution with a concentration of 1.0% is prepared to serve as an outer aqueous phase, 1.9 g of sodium chloride is added to the solution, and a pH value of the solution is adjusted to 10.0 by sodium hydroxide to prepare W. O is added to W, and mechanical stirring is carried out at a rotational speed of 300 rpm for 30 s to prepare a pre-emulsion. The pre-emulsion is poured into a rapid membrane emulsifier for emulsification and dispersion, where a used membrane tube has a pore size of 20 μm. The emulsion is solidified at a vacuum degree of −0.09 MPa and a temperature of 38 °C for 60 min. After the solidification is finished, washing and lyophilizing are carried out to obtain a donepezil sustained-release microsphere.

A particle size and particle size distribution determination method is used to determine that the prepared sustained-released microspheres have an average particle size of 14 μm and a Span value of 0.5. A high-performance liquid chromatography is used to determine a drug loading capacity and encapsulation efficiency of the prepared sustained-released microspheres, where the drug loading capacity is 59.9%, and the encapsulation efficiency is 99.8%.

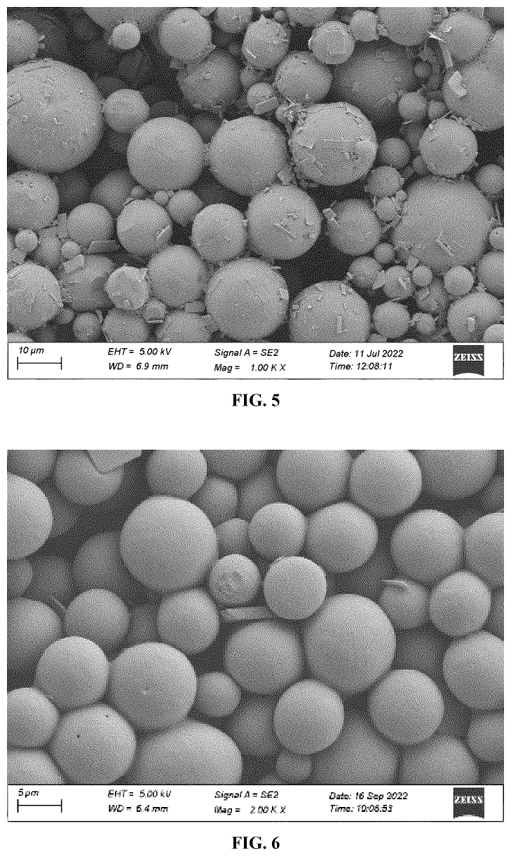

By comparing FIG. 5 and FIG. 6, it can be learned that the surface of the donepezil sustained-release microsphere prepared in Embodiment is relatively smooth and intact, with almost no depressions or cavities and favorable particle size uniformity, and a microsphere escape phenomenon outside the microsphere is significantly improved. At a high theoretical drug loading capacity of 60%, in comparison with Embodiment, it indicates that simultaneous addition of a pH regulator and an osmotic pressure regulator can not only significantly reduce crystal escape, but also render the surface of the microsphere smooth with almost no damage and pores.

### Embodiment 7

2.8 g of donepezil and 4 g of PLGA (LA:GA=75:25, intrinsic viscosity is from 0.6 to 0.7 dL/g) are weighed and then dissolved in 40 mL of dichloromethane to prepare O. 400 mL of polyvinyl alcohol aqueous solution with a concentration of 1.0% is prepared to serve as an outer aqueous phase, 1.5 g of sodium chloride is added to the solution, and a pH value of the solution is adjusted to 10.0 by sodium hydroxide to prepare W. O is added to W, and mechanical stirring is carried out at a rotational speed of 300 rpm for 30 s to prepare a pre-emulsion. The pre-emulsion is poured into a rapid membrane emulsifier for emulsification and dispersion, where a used membrane tube has a pore size of 20 μm. The emulsion is solidified at a vacuum degree of −0.09 MPa and a temperature of 38 °C for 60 min. After the solidification is finished, washing and lyophilizing are carried out to obtain a donepezil sustained-release microsphere.

A particle size and particle size distribution determination method is used to determine that the prepared sustained-released microspheres have an average particle size of 12 μm and a Span value of 0.6. A high-performance liquid chromatography is used to determine a drug loading capacity and encapsulation efficiency of the prepared sustained-released microspheres, where the drug loading capacity is 40.1%, and the encapsulation efficiency is 97.8%.

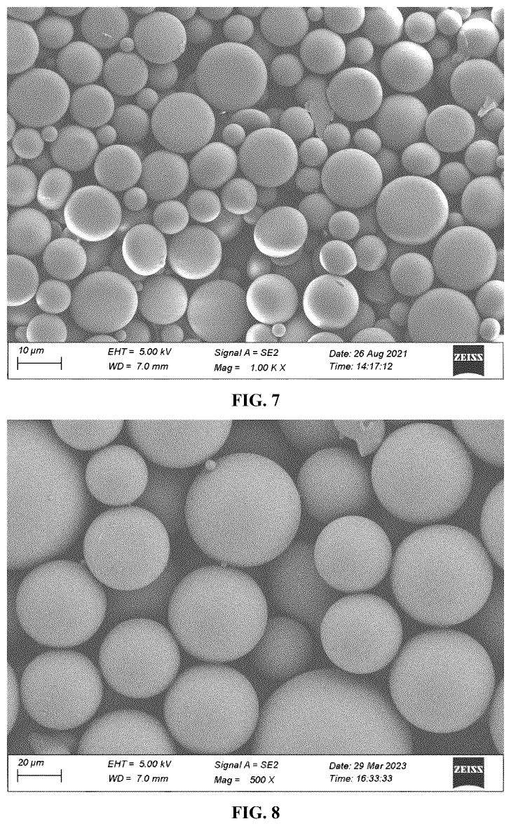

By comparing FIG. 6 and FIG. 7, it can be learned that the surface of the donepezil sustained-release microsphere prepared in Embodiment 7 is relatively smooth and intact, without depressions or cavities. However, there are a small number of microspheres with non-uniform particle size. This indicates that donepezil microsphere prepared after the drug loading capacity is reduced has no drug escape.

### Embodiment 8

2.8 g of donepezil and 4 g of PLGA (LA:GA=75:25, intrinsic viscosity is from 0.6 to 0.7 dL/g) are weighed and then dissolved in 40 mL of dichloromethane to prepare O. 400 mL of polyvinyl alcohol aqueous solution with a concentration of 1.0% is prepared to serve as an outer aqueous phase, 1.5 g of sodium chloride is added to the solution, and a pH value of the solution is adjusted to 10.0 by sodium hydroxide to prepare W. O is added to W, and mechanical stirring is carried out at a rotational speed of 300 rpm for 30 s to prepare a pre-emulsion. The pre-emulsion is poured into a rapid membrane emulsifier for emulsification and dispersion, where a used membrane tube has a pore size of 50 μm. The emulsion is solidified at a vacuum degree of −0.09 MPa and a temperature of 38 °C for 60 min. After the solidification is finished, washing and lyophilizing are carried out to obtain a donepezil sustained-release microsphere.

A particle size and particle size distribution determination method is used to determine that the prepared sustained-released microspheres have an average particle size of 28 μm and a Span value of 0.6. A high-performance liquid chromatography is used to determine a drug loading capacity and encapsulation efficiency of the prepared sustained-released microspheres, where the drug loading capacity is 40.9%, and the encapsulation efficiency is 99.8%.

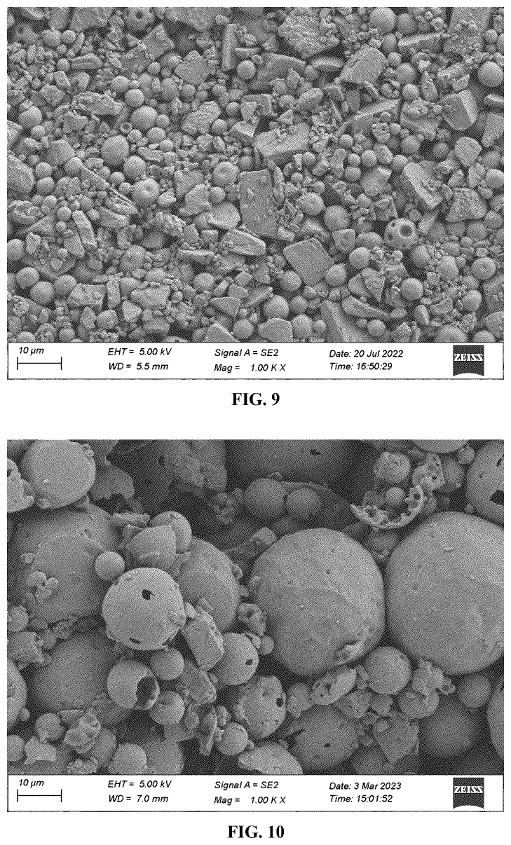

By comparing FIG. 7 and FIG. 8, it can be learned that the surface of the donepezil sustained-release microsphere prepared in Embodiment 7 is relatively smooth and intact, without depressions or cavities, and the particle size is relatively uniform. This indicates that in the present disclosure, appropriately increasing a membrane pore size during membrane emulsification has no significant effect on the particle size distribution of the microspheres.

### Comparative Example 1

1.0 g of donepezil and 1.0 g of PLGA (LA:GA=50:50, intrinsic viscosity is from 0.2 to 0.3 dL/g) are weighed and then dissolved in 10 mL of dichloromethane to prepare O. 100 mL of polyvinyl alcohol aqueous solution with a concentration of 1.0% is prepared to serve as an outer aqueous phase, and then W is prepared without pH adjustment. O is added to W, and mechanical stirring is carried out at a rotational speed of 300 rpm for 30 s to prepare a pre-emulsion. The pre-emulsion is poured into a rapid membrane emulsifier for emulsification and dispersion, where a used membrane tube has a pore size of 20 μm. The emulsion is solidified at a vacuum degree of −0.09 MPa and a temperature of 38 °C for 60 min. After the solidification is finished, washing and lyophilizing are carried out to obtain a donepezil sustained-release microsphere.

A particle size and particle size distribution determination method is used to determine that the prepared sustained-released microspheres have an average particle size of 13 μm and a Span value of 1.5. A high-performance liquid chromatography is used to determine a drug loading capacity and encapsulation efficiency of the prepared sustained-released microspheres, where the drug loading capacity is 38.1%, and the encapsulation efficiency is 76.2%.

By comparing FIG. 2, FIG. 3, FIG. 6, and FIG. 9, it can be learned that the donepezil sustained-release microsphere prepared in Comparative Example 1 exhibit extensive breakage, non-uniform particle size, rough surface morphology, severe collapse, and a large number of tiny pores, with drug escape and precipitation resulting in the formation of flake-like or block-like crystals. This indicates that the addition of the pH regulator and the osmotic pressure regulator in the present disclosure has significant impact on drug escape, the surface of the microsphere, and the drug loading capacity.

### Comparative Example 2

1.0 g of donepezil and 1.0 g of PLGA (LA:GA=50:50, intrinsic viscosity is from 0.2 to 0.3 dL/g) are weighed and then dissolved in 10 mL of dichloromethane to prepare O. 100 mL of polyvinyl alcohol aqueous solution with a concentration of 1.0% is prepared to serve as an outer aqueous phase, then 1.0 g of sodium chloride is added to the solution, and a pH value of the solution is adjusted to 10.0 by sodium hydroxide to prepare W. O is added to W, and mechanical stirring is carried out at a rotational speed of 300 rpm for 30 s to prepare a pre-emulsion. The pre-emulsion is poured into a rapid membrane emulsifier for emulsification and dispersion, where a used membrane tube has a pore size of 50 μm. The emulsion is solidified at a vacuum degree of −0.09 MPa and a temperature of 45 °C for 60 min. After the solidification is finished, washing and lyophilizing are carried out to obtain a donepezil sustained-release microsphere.

A particle size and particle size distribution determination method is used to determine that the prepared sustained-released microspheres have an average particle size of 13 μm and a Span value of 1.8. A high-performance liquid chromatography is used to determine a drug loading capacity and encapsulation efficiency of the prepared sustained-released microspheres, where the drug loading capacity is 24.2%, and the encapsulation efficiency is 48.4%.

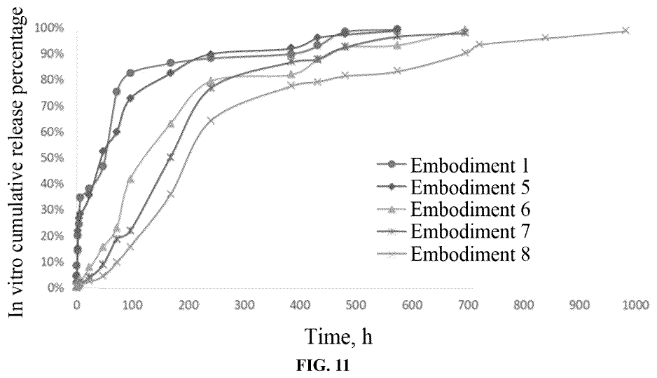

By comparing FIG. 6, and FIG. 10, it can be learned that the donepezil sustained-release microsphere prepared in Comparative Example 2 exhibits significant breakage, surface collapse, and non-uniform particle size, with drug escape and precipitation resulting in the formation of flake-like or block-like crystals. This indicates that when a temperature of the pressure-reduced volatilization for the microspheres is excessively high, the carrier material degrades under a high temperature, causing breakage and collapse of the microspheres. This ultimately results in ineffective drug encapsulation and a low drug loading capacity.

### Embodiment 11: In Vitro Cumulative Release Test

10 mg of microspheres prepared in Embodiment 1, Embodiment 5, Embodiment 6, Embodiment 7, and Embodiment 8, as well as PBS buffer (pH 7.4), are separately placed into 250 mL Erlenmeyer flasks, and then subjected to constant-temperature shaking at 37 °C in a water bath shaker. According to sampling time, 1 mL of the solution is pipetted from the Erlenmeyer flask, and an equal volume of PBS buffer is replenished, A collected solution is filtered through a 0.45 μm syringe filter, and 10 μL of the solution is tested by HPLC. In vitro cumulative release curves of the microspheres prepared in five embodiments are as shown in FIG. 11.

It can be seen from FIG. 11 that by controlling the pH of the aqueous phase and adjusting the osmotic pressure, drug escape can be effectively reduced, drug precipitation and crystallization can be avoided, leading to effective reduction of drug burst release. By increasing the particle size of the microsphere and appropriately reducing the drug loading capacity, the initial burst release of the drug can be effectively reduced, and the release cycle can be prolonged. The donepezil sustained-release microspheres prepared by the present disclosure has long-term sustained-release capability.

It should be noted that although the technical solution of the present disclosure is introduced with specific instances, those skilled in the art may understand that the present disclosure should not be limited thereto.

Various embodiments of the present disclosure have been described above. The foregoing description is illustrative, non-exhaustive, and not limited to the disclosed embodiments. Many modifications and variations are apparent to those skilled in the art without departing from the scope and spirit of the described embodiments. The selection of terms used herein is intended to best illustrate the principles of the embodiments, practical applications, or technical improvements in the industry, and to enable other persons skilled in the art to readily understand the embodiments disclosed herein.

1. A preparation method for a donepezil sustained-release microsphere, comprising the following steps:
mixing a surfactant, an osmotic pressure regulator, and water to obtain a mixture, adjusting a pH value of the mixture with a pH regulator to form an aqueous phase;
mixing donepezil, a carrier material, and an organic solvent to form an oil phase; and
dispersing the oil phase in the aqueous phase to form a pre-emulsion, carrying out membrane emulsification on the pre-emulsion to form an emulsion; finally solidifying, washing, and lyophilizing the emulsion to obtain the donepezil sustained-release microsphere.

2. The preparation method according to claim 1, wherein
the pre-emulsion is an oil-in-water pre-emulsion.

3. The preparation method according to claim 1, wherein the preparation method uses one or more substances selected from the group consisting of:
the surfactant comprises one or more components selected from the group consisting of polyvinyl alcohol, Tween, sodium dodecyl sulfonate, sodium dodecyl benzene sulfonate, and polyoxyethylene octylphenol ether, a weight ratio of the surfactant to donepezil is from 1:5 to 20:5;
the osmotic pressure regulator comprises one or more components selected from the group consisting of sodium chloride and potassium chloride; a weight ratio of the osmotic pressure regulator to donepezil is from 1:5 to 6:5;
the water comprises one or more components selected from the group consisting of ultra-pure water, double-distilled water, and water for injection; a volume ratio of the water to the organic solvent is from 5:1 to 50:1; and
the pH regulator comprises one or more components selected from the group consisting of hydrogen chloride and sodium hydroxide; the pH regulator is used to adjust a pH value of the mixture to from 6.0 to 12.0.

4. The preparation method according to claim 1, wherein the preparation method uses one or more substances selected from the group consisting of:
the carrier material is a biodegradable carrier material, the carrier material comprises one or more components selected from the group consisting of poly(lactic-co-glycolic acid), poly(lactic acid)-poly(ethylene glycol) copolymer, and polylactic acid, the carrier material has intrinsic viscosity from 0.15 dL/g to 0.90 dL/g; a weight ratio of the carrier material to donepezil is from 2:3 to 12:3; and
the organic solvent comprises one or more members selected from the group consisting of trichloromethane, dichloromethane, ethyl acetate, acetone, acetonitrile, and absolute ethyl alcohol; a dosage ratio of the organic solvent to donepezil is from 4 to 20 mL:1 g.

5. The preparation method according claim 1, wherein the preparation method uses one or more substances selected from the group consisting of:
the dispersion is accomplished by one or more means selected from the group consisting of stirring and ultrasonication; the mechanical stirring lasts for 30 to 60 s; the mechanical stirring is carried out at a rotational speed of 250 to 350 rpm;
the emulsification is accomplished by means of membrane emulsification; and
the solidification is accomplished by volatilization or evaporation; the pressure-reduced volatilization is carried out at a vacuum degree of −0.09 to −0.05 MPa; the pressure-reduced volatilization is carried out at 20 to 42 °C for 20 to 300 min.

6. The preparation method according to any one of claim 1, wherein
the preparation method further comprises the following steps:
solidifying the emulsion, followed by washing and lyophilizing.

7. The preparation method according to any one of claim 1, wherein the preparation method uses one or more substances selected from the group consisting of:
the donepezil sustained-release microsphere has a particle size of 10 to 50 μm;
encapsulation efficiency of the donepezil sustained-release microsphere is over 80%; and
a drug loading capacity of the donepezil sustained-release microsphere is 20% to 60%.

8. The preparation method according to any one of claim 3, wherein the weight ratio of the surfactant to donepezil is from 4:5 to 8:5.

9. The preparation method according to any one of claim 3, wherein the weight ratio of the osmotic pressure regulator to donepezil is from 1.5:5 to 4:5.

10. A donepezil sustained-release microsphere, wherein the sustained-release microsphere is prepared by a preparation method comprising:
mixing a surfactant, an osmotic pressure regulator, and water to obtain a mixture, adjusting a pH value of the mixture with a pH regulator to form an aqueous phase;
mixing donepezil, a carrier material, and an organic solvent to form an oil phase; and
dispersing the oil phase in the aqueous phase to form a pre-emulsion, carrying out membrane emulsification on the pre-emulsion to form an emulsion; finally solidifying, washing, and lyophilizing the emulsion to obtain the donepezil sustained-release microsphere.

11. The donepezil sustained-release microsphere according to claim 10, wherein the sustained-release microsphere is prepared by a preparation method comprising: the pre-emulsion is an oil-in-water pre-emulsion.

12. The donepezil sustained-release microsphere according to claim 10, wherein the preparation method uses one or more substances selected from the group consisting of:
the surfactant comprises one or more components selected from the group consisting of polyvinyl alcohol, Tween, sodium dodecyl sulfonate, sodium dodecyl benzene sulfonate, and polyoxyethylene octylphenol ether, a weight ratio of the surfactant to donepezil is from 1:5 to 20:5;
the osmotic pressure regulator comprises one or more components selected from the group consisting of sodium chloride and potassium chloride; a weight ratio of the osmotic pressure regulator to donepezil is from 1:5 to 6:5;
the water comprises one or more components selected from the group consisting of ultra-pure water, double-distilled water, and water for injection; a volume ratio of the water to the organic solvent is from 5:1 to 50:1; and
the pH regulator comprises one or more components selected from the group consisting of hydrogen chloride and sodium hydroxide; the pH regulator is used to adjust a pH value of the mixture to from 6.0 to 12.0.

13. The donepezil sustained-release microsphere according to claim 10, wherein the preparation method uses one or more substances selected from the group consisting of:
the carrier material is a biodegradable carrier material, the carrier material comprises one or more components selected from the group consisting of poly(lactic-co-glycolic acid), poly(lactic acid)-poly(ethylene glycol) copolymer, and polylactic acid, the carrier material has intrinsic viscosity from 0.15 dL/g to 0.90 dL/g; a weight ratio of the carrier material to donepezil is from 2:3 to 12:3; and
the organic solvent comprises one or more components selected from the group consisting of trichloromethane, dichloromethane, ethyl acetate, acetone, acetonitrile, and absolute ethyl alcohol; a dosage ratio of the organic solvent to donepezil is from 4 to 20 mL:1 g.

14. The donepezil sustained-release microsphere according to claim 10, wherein the preparation method uses one or more substances selected from the group consisting of:
the dispersion is accomplished by one or more means selected from stirring and ultrasonication, the mechanical stirring lasts for 30 to 60 s; the mechanical stirring is carried out at a rotational speed of 250 to 350 rpm;
the emulsification is accomplished by means of membrane emulsification; and
the solidification is accomplished by volatilization or evaporation, the pressure-reduced volatilization is carried out at a vacuum degree of −0.09 to −0.05 MPa; the pressure-reduced volatilization is carried out at 20 to 42 °C for 20 to 300 min.

15. The donepezil sustained-release microsphere according to claim 10, wherein
the preparation method further comprises the following steps: solidifying the emulsion, followed by washing and lyophilizing.

16. The donepezil sustained-release microsphere according to claim 10, wherein the preparation method uses one or more substances selected from the group consisting of:
the donepezil sustained-release microsphere has a particle size of 10 to 50 μm;
encapsulation efficiency of the donepezil sustained-release microsphere is over 80%; and
a drug loading capacity of the donepezil sustained-release microsphere is 20% to 60%.

17. A pharmaceutical formulation of donepezil, wherein the pharmaceutical formulation comprises the sustained-release microsphere according to claim 8.

18. The pharmaceutical formulation of donepezil according to claim 17, wherein the pharmaceutical formulation further comprises at least one pharmaceutically acceptable excipient.

19. The pharmaceutical formulation of donepezil according to claim 17, wherein the pharmaceutical formulation is a sustained-release formulation.

20. The pharmaceutical formulation of donepezil according to claim 17, wherein the pharmaceutical formulation is an injection.
